(19) United States
(12) Patent Application Publication
(10) Pub. No.: US 2022/0062491 A1
(43) Pub. Date: Mar. 3, 2022

(54) AIR TREATMENT SYSTEM WITH AIRFLOW DETECTION

(71) Applicants: Roger Stull, Des Moines, WA (US); Linda Hansen-Stull, Des Moines, WA (US); Rikel K. Getty, Des Moines, WA (US); Akos Bohm, Des Moines, WA (US); Nelson Hall, Des Moines, WA (US)

(72) Inventors: Roger Stull, Des Moines, WA (US); Linda Hansen-Stull, Des Moines, WA (US); Rikel K. Getty, Des Moines, WA (US); Akos Bohm, Des Moines, WA (US); Nelson Hall, Des Moines, WA (US)

(73) Assignee: EnspiAIR LLC, Des Moines, WA (US)

(21) Appl. No.: 17/465,070

(22) Filed: Sep. 2, 2021

Related U.S. Application Data
(60) Provisional application No. 63/224,461, filed on Jul. 22, 2021.
(60) Provisional application No. 63/073,734, filed on Sep. 2, 2020.

(51) Int. Cl. A61L 9/20

(52) U.S. Cl. CPC A61L 9/205; A61L 2209/16; A61L 2209/12; A61L 2209/111

(57) ABSTRACT

An air treatment system, such as a germicidal ultraviolet air treatment fixture, for use in vehicles. The air treatment system may include an airflow detection circuit for selectively energizing an ultraviolet light source based on detection of air movement. The air treatment system may also include a Photo Catalytic Oxidation (PCO) process. The ultraviolent light source and/or a photo catalyst may be installed in, for example, HVAC ducting, etc.

### CROSS-REFERENCES TO RELATED APPLICATIONS

This application claims benefit of U.S. Provisional Application No. 63/073,734, filed Sep. 2, 2020, and U.S. Provisional Application No. 63/224,461, filed Jul. 22, 2021, the disclosures of which are incorporated herein in their entirety.

### FIELD OF THE DISCLOSURE

The present disclosure relates generally to air treatment equipment, and in some examples, to a germicidal ultraviolet air treatment fixture for vehicles. In examples of the air treatment fixture, an airflow detection circuit is provided, for selectively energizing the ultraviolet light source based on detection of air movement.

### BACKGROUND

Recent concerns about the spread of the Corona virus have raised awareness of how disease pathogens are spread. The enclosed environment within a transportation vehicle has been a particular target of concern for public health professionals. Theoretically, a highly contagious airborne pathogen could spread from one infected passenger to everyone on board, due to the enclosed environment and the constant circulation of air within the cabin.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with an aspect of the present disclosure, an air treatment system is provided. In an embodiment, the air treatment system comprises an ultraviolet (UV)-C light source and an air movement detection circuit for detecting air movement. The air movement detection circuit is configured to supply power to the ultraviolet (UV)-C light source based on detection of air movement.

In any embodiment, the air movement detection circuit comprises an air movement sensor electrically connected to an ultraviolet (UV)-C light source supply switch. The air movement sensor is configured to output a signal based on detection of air movement.

In any embodiment, the ultraviolet (UV)-C light source supply switch is configured to switch from an OFF state to an ON state from receipt of the signal from the air movement sensor.

In any embodiment, the ultraviolet (UV)-C light source supply switch is connected in electrical communication with a power source, and wherein the ultraviolet (UV)-C light source receives power when the ultraviolet (UV)-C light source supply switch is in the ON state.

In any embodiment, the air movement sensor includes an anemometer.

In any embodiment, the system further includes a duct having at least one interior surface, wherein the ultraviolet (UV)-C light source is mounted in the duct adjacent the at least one interior surface.

In any embodiment, the air treatment system further comprises a photo catalyst layer disposed on the interior surface.

In any embodiment, the air treatment system further comprises a surface coated with a photo catalyst, the surface disposed adjacent the ultraviolet (UV)-C light source.

In accordance with another aspect of the present disclosure, a vehicle is provided, which comprises any of the embodiments of the air treatment system described herein.

In accordance with another aspect of the present disclosure, an air treatment system is provided. In an embodiment, the air treatment system comprises a duct having at least one interior surface, an ultraviolet (UV)-C light source mountable in the duct adjacent the at least one interior surface, and a photo catalyst layer disposed on the interior surface.

In any embodiment, the air treatment system further comprises further comprises an air movement detection circuit for detecting air movement in the duct. The air movement detection circuit is configured to supply power to the ultraviolet (UV)-C light source based on detection of air movement.

In any embodiment, the air movement detection circuit comprises an air movement sensor electrically connected to an ultraviolet (UV)-C light source supply switch. The air movement sensor is configured to output a signal based on detection of air movement. The ultraviolet (UV)-C light source supply switch is configured to switch from an OFF state to an ON state based on the signal from the air movement sensor.

In any embodiment, the ultraviolet (UV)-C light source supply switch is connected in electrical communication with a power source. The ultraviolet (UV)-C light source receives power when the ultraviolet (UV)-C light source supply switch is in the ON state.

In any embodiment, the air treatment system further comprises a heat sensor configured to sense the temperature of the ultraviolet (UV)-C light source.

In any embodiment, the air treatment system further comprises an indicator light configured to indicate the operational state of the ultraviolet (UV)-C light source.

In accordance with another aspect of the present disclosure, a method is provided of disinfecting air to be discharged into a vehicle cabin of a vehicle. The vehicle having one or more air vents in gas communication with the vehicle cabin and an HVAC system including a plurality of ducts and a blower. In an embodiment, the method comprises circulating air through at least duct of the plurality of ducts, exposing the air to ultraviolet (UV)-C light, and thereafter discharging the air into the vehicle cabin.

In any embodiment of the method, circulating air through at least duct of the plurality of ducts includes drawing air into the at least one duct by vehicle movement or by the blower of the HVAC system.

In any embodiment, the method further comprises sensing air circulating through the at least one duct and supplying power to an ultraviolet (UV)-C light source based on the sensing air circulating through the at least one duct. The ultraviolet (UV)-C light source emits the ultraviolet (UV)-C light and exposes the air to the ultraviolet (UV)-C light.

In any embodiment of the method, circulating air through at least duct of the plurality of ducts includes drawing air into the at least one duct by operation of the blower of the HVAC system. In these embodiments, the method further comprises determining the operational state of the blower, and selectively supplying power to an ultraviolet (UV)-C light source based on the determining the operational state of the blower.

In any embodiment, the method further comprises sensing the temperature of an ultraviolet (UV)-C light source, the air being exposed by the ultraviolet (UV)-C light generated by the ultraviolet (UV)-C light source, and selectively supplying power to the ultraviolet (UV)-C light source based on the temperature of the ultraviolet (UV)-C light.

### DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1 is a schematic representation of an air treatment system in accordance with an embodiment of the disclosure;

FIG. 2 is a schematic representation of an environment in which an air treatment system in accordance with an embodiment of the disclosure can be employed;

FIG. 3 is a circuit diagram of one representative embodiment of the air treatment system in accordance with an aspect of the disclosure;

FIG. 4 is a partial view of a duct, into which a UV light source of the air treatment system is mounted.

### DETAILED DESCRIPTION

The present disclosure includes examples of methodologies and technologies that relate to air sterilization using UV-C light. It is well understood that light in the UV-C range (approx. 200 nm to 280 nm) inactivates and kills bacteria, molds, protozoa, viruses and yeasts.

In embodiments disclosed herein, one or more treatment systems are described for installation in, for example, a Heating Ventilation and Air Conditioning (HVAC) system having at least duct, HVAC ducting (e.g., a plurality of ducts), HVAC ductwork, etc. These treatment systems include a source of ultraviolet (UV)-C light, such as a UV-C lamp, in light communication with the HVAC ducting, etc., through which air passes or is contained. In use, the UV-C light is used to decontaminate air passing through or contained in the ducting, etc.

According to an optional embodiment, the system is automatically activated (e.g., energized, turned on, etc.) by an air movement sensor. In use, the air movement sensor senses air flow within the HVAC ducting, etc., and activates an associated switch to supply power to the source of UV-C light, such as the UV-C lamp.

The air movement sensor may be, for example, an anemometer (e.g., hot wire anemometer), a solid state sensor, etc. In some embodiments, the air movement sensor does not involve any moving mechanical parts. In combining the UV-C lamp with air movement ON-OFF switching capabilities, no outside trigger or switching is required for operation. This is advantageous in a vehicle application because air flow may occur with or without the blower motor running. Additionally, in a vehicle environment, the UV-C lamp should not be left in its light emitting state at all times since it could drain the battery source (vehicle) or possibly overheat. It would also shorten the service life of the lamp. The air movement sensor may also serve as a safety interlock. Should the system be removed from the HVAC, airflow would be interrupted and the unit would turn the lamp off.

In a vehicle application, it is desirable to activate the UV-C lamp only when air is flowing through the HVAC system. In this application, air may be flowing with or without the HVAC blower motor running. For example, air can flow as a result of automobile movement, without the aid of a blower. In this case, using the blower motor power as a signal to energize the UV-C lamp is inadequate in some embodiments.

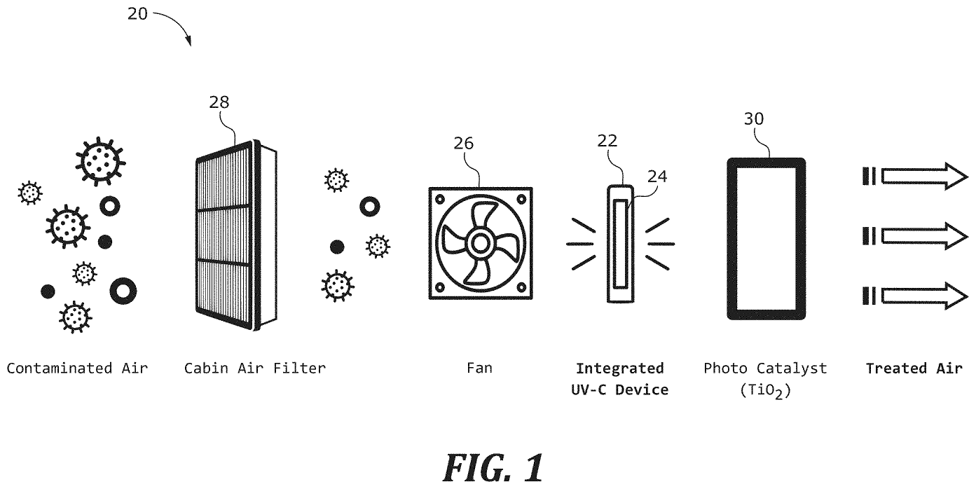

FIG. 1 is a schematic representation of an air treatment system, generally designated 20, formed in accordance with an embodiment of the disclosure. Generally described, the air treatment system includes a UV-C device 22 containing or associated with a UV-C light source 24, a blower or fan 26, an optional air filter 28 and an optional photocatalyst 30.

As shown in FIG. 1, contaminated air traverses through, for example, ducting of an HVAC system. In an embodiment, the contaminated air is blown or pulled through the ducting via the blower or fan 26 or the like. In vehicle applications, contaminated air may circulate through the ducting based on, for example, vehicle movement.

Once passing or circulating through the ducting, the contaminated air is exposed to the UV-C light source 24 of the UV-C device 22. Exposure to the UV-C light source 24 disinfects the air, leaving germicidal-free air to exit the ducting.

In some embodiments, one or more air filters 28 (optional) can be employed to filter the contaminated air upstream of the UV-C light source 24 or filter the treated air downstream of the UV-C light source 24. In the system shown in FIG. 1, the air filter 28 is located upstream of the UV-C light source 24. Additionally or alternatively, an optional photo catalyst, such as titanium dioxide, can be coated onto interior surfaces of a duct adjacent the UV-C light source 24. As used herein, the term “treated air” includes air that has been exposed to UV-C light and/or subject to a Photo Catalytic Oxidation (PCO) process.

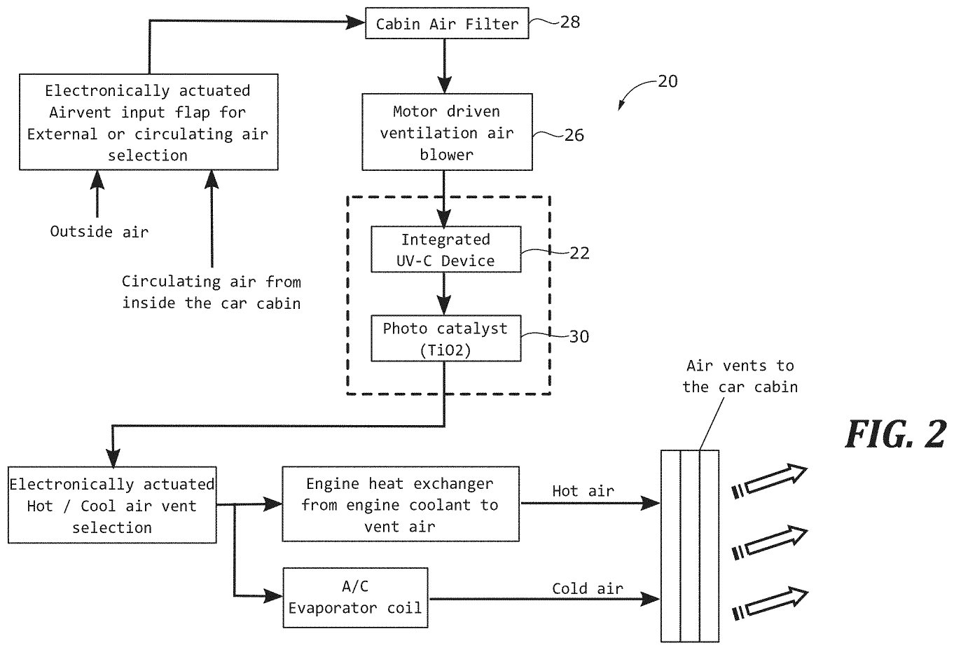

FIG. 2 is a schematic representation of a representative environment, such as vehicle, in which embodiments of the air treatment system, such as the air treatment system 20, can be employed. Generally described, in the environment shown, air can enter the air treatment system via either outside air (vent mode) or recirculating air (recirculation mode) from the vehicle. A selector switch or switches of the vehicle's HVAC system can be employed for selection purposes. Upon operation of the selector switch(es), an air vent input flap or the like is actuated for selection of either the vent mode or the recirculating mode.

In the embodiment shown, the contaminated air is blown or pulled through the ducting via a blower or fan 26, such as the existing blower, fan, etc., of the vehicle. In other embodiments, contaminated air circulates through the ducting based on vehicle movement, etc.

Once circulating through the ducting, the contaminated air is exposed to an UV-C light source, such as the UV-C light source 24 of FIG. 1. Exposure to the UV-C light source of the UV-C device 22 disinfects the air, leaving germicidal-free air to exit the ducting.

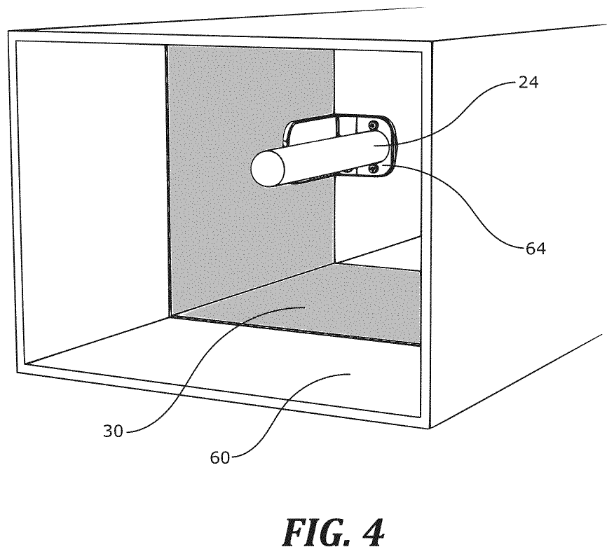

In some embodiments, one or more air filters 28 can be employed to filter the contaminated air upstream of the UV-C light source, as shown, or filter the treated air downstream of the UV-C device 22. In the system shown in FIG. 2, the air filter 28 is located upstream of the UV-C device 22. An optional photo catalyst 30, such as titanium dioxide, may be coated onto interior surfaces of a duct (see FIG. 4) adjacent the UV-C light source of the UV-C device 22. In use, light form the UV-C light source impinges onto the photo catalyst 30 and a Photo Catalytic Oxidation (PCO) process occurs.

The treated air is then discharged into the vehicle cabin via the one or more air vents. Prior to exiting the air vents, the treated air travels downstream of the UV-C light source and is exposed to a heat exchanger for either cooling the treated air or heating the treated air. Selection of either heating or cooling the treated air is accomplished, for example, via a hot/cold air selector. Once selected, the treated air is routed past the selected heat exchanger to either heat or cool the treated air.

When the outside air is being distributed throughout the vehicle's cabin in vent mode, the air treatment system 20 with the optional titanium dioxide (TiO₂) photo catalyst 30 removes odors and volatile organic compounds. In recirculation mode, the cabin air is disinfected while cycling through the air treatment system 20. Longer operation times in recirculation mode provides cleaner air quality with decreased viral existence.

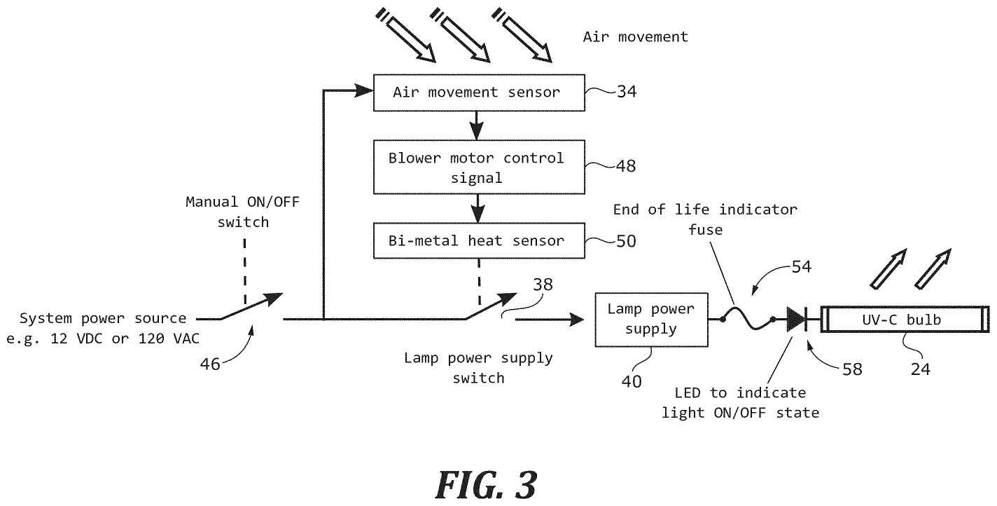

Turning now to FIG. 3, one or more components of the air treatment system 20 will be described in more detail. FIG. 3 is a representative circuit diagram comprising one or more components of the air treatment system 20 in accordance with an aspect of the disclosure. As shown in FIG. 3, the air treatment system 20 comprises a UV-C light source 24, such as an UV-C lamp, and an optional air movement detection circuit that selectively connects the UV-C light source 24 in electrical communication to a power source, such as a battery, mains power, etc. In some embodiments, the air treatment system 20 also includes a light source power supply 40 that processes or converts the supply of power from the battery, mains power, etc., into power suitable for use by the light source 24.

In the embodiment shown, the optional air movement detection circuit includes an air movement sensor 34 and a light source power supply switch 38. In an embodiment, the air movement sensor is configured detect air movement, and if present, to generate a signal indicative of air movement. In the air movement detection circuit, the air movement sensor 34 is connected in electrical communication with the UV-C light source power supply switch 38 or lamp power supply switch. In an embodiment, the UV-C light source power supply switch 38 includes one or more relays to carry out at least some of the switching capabilities.

For detecting air movement or flow, the air movement sensor 34 (e.g., detector) can be or include, for example, an anemometer, a solid state sensor, etc. In some embodiments, the anemometer can be a hot-element or hot-wire anemometer type sensor, which is calibrated to energize the UV-C light source when a minimum specified airflow is detected. This hot element, for example, could be a discrete wire or could be a trace on a printed circuit board. When air flows over the hot element, it cools and its electrical resistance drops. The resistance is constantly monitored and when low enough, the output signal generated by the detector triggers the UV-C light source power supply switch 38 to an ON state and turns on the UV-C light source 24. Other types of anemometer type sensors can be used, including anemometers of the laser doppler type, of the ultrasonic type, acoustic resonance type, etc. In some embodiments, the hot wire anemometer is a constant current anemometer or a constant voltage anemometer.

The UV-C light source power supply switch 38 is activated (switched to the ON position, allows power to flow through the switch, etc.) upon receipt of the signal generated by the air movement sensor 34 (e.g., when the air movement sensor detects air movement, such as a preselected amount of air movement.) When in the ON state, system power is delivered to the UV-C light source power supply 40 which in turn energizes the UV-C lamp 24. Once air movement ceases or drops below a preselected amount, the UV-C light source power supply switch 38 transitions to an OFF state, cutting off system power to the UV-C light source.

The UV-C light source 24 can be any light source that is capable of emitting UV-C light. For example, in some embodiments the UV-C light source is a Low Pressure UltraViolet (LPUV) lamp. A LPUV lamp is a low dose mercury vapor lamp. Similar to a neon lamp, it is constructed using a clear envelope, with the envelope material being quartz instead of glass. The envelope is filled with an inert gas and a small amount of mercury. Since common glass blocks UV-C light, the lamp envelope is replaced with quartz to allow the UV-C light to escape. Excitation voltage of the LPUV lamp is typically 300V to over 1000V, although other voltages may be used. Light output power is typically 6 watts to several hundred watts, although other wattages may be used depending on its intended application (e.g., quantity of airflow (cfm).

In other embodiments, a UV-C Light Emitting Diode (LED) can be employed. A UV-C LED is a specifically designed LED that emits light in the UV-C range. Excitation voltage for the UV-C LED is a few volts. Light output power level is a few milliwatts in some embodiments. Embodiments of the air treatment system may employ a number of UV-C LEDs to match or exceed desired air treatment protocols.

As shown in FIG. 3, the UV-C light source 24 is selectively connected to a power source via the airflow detection circuit. The power source can be one or more batteries, ultracapacitors, alternators or other energy sources, for use in vehicle application, or mains power, generators, etc., for use in stationary environments. As briefly described above, power supplied by the power source is fed via the air movement detection circuit to the optional power supply 40, which is configured to convert power to match or exceed the excitation requirements (e.g., voltage/current) of the UV-C light source 24.

Additional features, although optional, can be employed by the air treatment system 20. For example, as shown in the circuit diagram of FIG. 3, the air treatment system 20 may also include an optional on/off switch 46 to enable the user to control when activation or deactivation of the UVC light source occurs. In some embodiments, the optional air movement sensor 34 ensures that the system is running automatically in the presence of any kind of air flow. In some embodiments, one or more circuits, such as the circuit containing the UV-C light source power supply switch 38, may receive a blower motor control signal 48 in case the air movement sensor 34 fails to operate. This ensures that the UVC light source 24 is turned on whenever the users operates the heating and ventilation system of the automobile (i.e., when the HVAC system of the vehicle is pulling air through the air treatment system.)

In some embodiments, an optional heat sensor 50 may be provided to ensure safe operational temperatures of the UV-C light source 24. In operation, one or more circuits of the air treatment system, such as the circuit containing the UV-C light source power supply switch 38, are configured to receive signals from the heat sensor 50 and to shut off the UV-C light source 24 (e.g., shut of supply of power to the light source) in case the temperature rises above a threshold level, such as above 85 degrees Celsius. For example, the UV-C light source power supply switch 38 and/or associated circuitry may be configured to transition from the ON state of the switch to the OFF state of the switch.

Other safety features may be employed, such as a fuse 54. For example, studies have shown that as the UV-C lamp ages over time, its germicidal effectiveness is reduced. The optional fuse 54 is configured to break the electronic circuit as the bulb is “overdrawing” current towards the end of its operational life. In some UV-C lamps, this occurs approximately after 4,000 hours of usage.

The air treatment system 20 may further include an optional indicator light 58 visible to the user. For example, a LED indicator light may be used to inform the user that the air treatment system is operational and discharging treated air. In some embodiments, the LED indicator light can be used to inform (e.g., switches color) the user of UV-C bulb replacement maintenance when the previously described fuse breaks the circuit. This ensures (for safety purposes) the user or the maintenance technician does not look at or disturb the lamp which is protected inside the vehicle's ducting.

FIG. 4 is a schematic view of the UV-C light source 24 being mounted within a duct of, for example, an HVAC system of a vehicle. As shown in the FIG. 4, the UV-C light source 24 is mounted inside duct 60 of a vehicle via a mounting bracket 64. As further shown in FIG. 4, interior surfaces of the duct 60 surrounding the UV-C light source 24 are coated with, for example, a photo catalyst 30, such as titanium dioxide (TiO2). For example, in the embodiment shown in FIG. 4, the photo catalyst 30 is coated on the four interior walls of the (rectangular) duct 60. Accordingly, the air treatment system 20 may optionally incorporate a Photo Catalytic Oxidation (PCO) process by which volatile organic compounds (VOCs), bacteria, mold & fungus are destroyed by incorporating photon and ultraviolet (UV) energy.

In the embodiment shown, the vicinity of the UVC light source, for example, adjacent walls, are coated with titanium dioxide. In use, the UV-C light source shines light onto the photo catalyst coating, thereby producing hydroxyl radicals which breakdown organic compounds thus reducing fungal, bacterial and viral microorganisms. By using a photo catalyst coating, the PCO process does not alter airflow of the vehicle's HVAC system.

In some embodiments, the mounting bracket 64 is specifically designed to enable a technician to install the UV-C light source and associated components in a simple manner. In an embodiment, the mounting bracket provides support for the UV-C light source 24 (e.g., UV-C lamp or bulb), the optional air movement detection circuit, and the optional heat sensor.

It should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Moreover, some of the method steps can be carried serially or in parallel, or in any order unless specifically expressed or understood in the context of other method steps.

In the foregoing description, specific details are set forth to provide a thorough understanding of exemplary embodiments of the present disclosure. It will be apparent to one skilled in the art, however, that the embodiments disclosed herein may be practiced without embodying all of the specific details. In some instances, well-known method/process steps have not been described in detail in order not to unnecessarily obscure various aspects of the present disclosure. Further, it will be appreciated that embodiments of the present disclosure may employ any combination of features described herein.

Throughout this specification, terms of art may be used. These terms are to take on their ordinary meaning in the art from which they come, unless specifically defined herein or the context of their use would clearly suggest otherwise.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

The embodiments of the invention in which an exclusive property or privilege is claimed are defined as follows:

1. An air treatment system, comprising:
an ultraviolet (UV)-C light source;
an air movement detection circuit for detecting air movement, the air movement detection circuit configured to supply power to the ultraviolet (UV)-C light source based on detection of air movement.

2. The air movement system of claim 1, wherein the air movement detection circuit comprises an air movement sensor electrically connected to an ultraviolet (UV)-C light source supply switch, wherein the air movement sensor is configured to output a signal based on detection of air movement.

3. The air movement system of claim 2, wherein the ultraviolet (UV)-C light source supply switch is configured to switch from an OFF state to an ON state from receipt of the signal from the air movement sensor.

4. The air movement system of claim 3, wherein the ultraviolet (UV)-C light source supply switch is connected in electrical communication with a power source, and wherein the ultraviolet (UV)-C light source receives power when the ultraviolet (UV)-C light source supply switch is in the ON state.

5. The air movement system of claim 2, wherein the air movement sensor includes an anemometer.

6. The air movement system of claim 1, further including a section of a duct having at least one interior surface, wherein the ultraviolet (UV)-C light source is mounted in the section of the duct and adjacent the at least one interior surface.

7. The air movement system of claim 1, further comprising a photo catalyst layer disposed on the interior surface.

8. The system of claim 1, further comprising a surface coated with a photo catalyst, the surface disposed adjacent the ultraviolet (UV)-C light source.

9. A vehicle comprising the air treatment system of claim 8.

10. An air treatment system, comprising:
a duct having at least one interior surface;
an ultraviolet (UV)-C light source mountable in the duct and adjacent the at least one interior surface; and
a photo catalyst layer disposed on the interior surface.

11. The air treatment system of claim 10, further comprising an air movement detection circuit for detecting air movement in the duct, the air movement detection circuit configured to supply power to the ultraviolet (UV)-C light source based on detection of air movement.

12. The air treatment system of claim 11, wherein the air movement detection circuit comprises an air movement sensor electrically connected to an ultraviolet (UV)-C light source supply switch, wherein the air movement sensor is configured to output a signal based on detection of air movement, and wherein the ultraviolet (UV)-C light source supply switch is configured to switch from an OFF state to an ON state based on the signal from the air movement sensor.

13. The air movement system of claim 12, wherein the ultraviolet (UV)-C light source supply switch is connected in electrical communication with a power source, and wherein the ultraviolet (UV)-C light source receives power when the ultraviolet (UV)-C light source supply switch is in the ON state.

14. The system of claim 10, further comprising a heat sensor configured to sense the temperature of the ultraviolet (UV)-C light source.

15. The system of claim 10, further comprising an indicator light configured to indicate the operational state of the ultraviolet (UV)-C light source.

16. A method of disinfecting air to be discharged into a vehicle cabin of a vehicle, the vehicle having one or more air vents in gas communication with the vehicle cabin and an HVAC system including a plurality of ducts and a blower, the method comprising:
circulating air through at least duct of the plurality of ducts;
exposing the air to ultraviolet (UV)-C light; and thereafter
discharging the air into the vehicle cabin.

17. The method of claim 16, wherein said circulating air through at least duct of the plurality of ducts includes drawing air into the at least one duct by vehicle movement or by the blower of the HVAC system.

18. The method of claim 16, further comprising
sensing air circulating through the at least one duct; and
supplying power to an ultraviolet (UV)-C light source based on said sensing air circulating through the at least one duct,
wherein the ultraviolet (UV)-C light source emits the ultraviolet (UV)-C light and exposes the air to said ultraviolet (UV)-C light.

19. The method of claim 17, wherein said circulating air through at least duct of the plurality of ducts includes drawing air into the at least one duct by operation of the blower of the HVAC system, the method further comprising
determining the operational state of the blower; and
selectively supplying power to an ultraviolet (UV)-C light source based on said determining the operational state of the blower.

20. The method of claim 16, further comprising
sensing the temperature of an ultraviolet (UV)-C light source, the air being exposed by the ultraviolet (UV)-C light generated by the ultraviolet (UV)-C light source; and
selectively supplying power to the ultraviolet (UV)-C light source based on said temperature of the ultraviolet (UV)-C light.
